SEQUENCE LISTING (279 sequences)

SEQ ID NO: 1 - protein, 214 aa, Homo Sapiens
DIQMTQSPSSLSASVGDRVTITCRASQSISTYLNWYQQKPGKAPKFLIYAASSLQSGVPSRFSGSGSGTDFTLTISSLQPEDFAAYYCQQSHSAPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, Homo Sapiens
QVQLQESGPGLVKPSQTLSLNCTVSGGSISSGGYYWSWIRQHPGKGLEWIGYIHYSGSTYYNPSLKSRITISADTSKNQFSLKLNSVTAADTAVYYCARDRGGGDYGRMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 112 aa, Homo Sapiens
DVVMTQSPLSLPVTLGQPASISCRSSQSLVYSDGNTYLNWFQQRPGQSPRRLIYKVSNWDSGVPDRFNGSGSGTDFTLKISRVEAEDVGVYYCMQSTHWPITFGQGTRLEIK

SEQ ID NO: 4 - protein, 112 aa, Homo Sapiens
DVVMTQSPLSLPVTLGQPASISCRSSQSLVYSDGNTYLNWFQQRPGQSPRRLIYKVSNWDSGVPDRFSGSGSGTDFTLKISRVEAEDVGVYYCIQGTHWPTTFGQGTRLEIK

SEQ ID NO: 5 - protein, 112 aa, Homo Sapiens
DVVMTQSPLSLPVTLGQPASISCRSSQSLVYSDGNTYLNWLQQRPGQSPRRLIYKVSNWDSGVPDRFSGSGSGTDFTLKISRVEAEDVGVYYCMQGTHWPITFGQGTRLEIK

SEQ ID NO: 6 - protein, 112 aa, Homo Sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQGIQLPCSFGQGTKLEIK

SEQ ID NO: 7 - protein, 112 aa, Homo Sapiens
DIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQSIQLPLTFGGGTKVEIK

SEQ ID NO: 8 - protein, 112 aa, Homo Sapiens
DIVMTQTPLSLSVTPGQPASISCKSSQSLLHSDGKTYLYWFLQKPGQPPQPLIYEVSNRFSGVPDRFSGSGSGTDFTLKISRVEAEDVGVYYCMQSIQLPITFGHGTRLEIK

SEQ ID NO: 9 - protein, 107 aa, Homo Sapiens
DIQMTQSPSSLSASVGDRVTITCRASQGIANYLAWYQQKPGKVPKLLIYVASTLQSGVPSRFSGSGSGTDFTLTISSLQPEDVATYYCQNYNSAPFTFGPGTKVDIK

SEQ ID NO: 10 - protein, 107 aa, Homo Sapiens
DIQMTQSPSSLSASVGDRVTIICRASQGISNDLAWYQQKPGKVPKLLIYAASTLQSGVPSRFSGSGSGTDFTLTISSLQPEDVATYYCQKYNSVPLTFGGGTKVEIK

SEQ ID NO: 11 - protein, 112 aa, Homo Sapiens
NIVMTQTPLSSPVTLGQPASISCRSSQSLVHSDGNTYLSWLQQRPGQPPRLLIYKISNRFSGVPDRFSGSGAGTDFTLKISRVEAEDVGVYYCMQATQFPHTFGPGTKVDIK

SEQ ID NO: 12 - protein, 112 aa, Homo Sapiens
EIVMTQTPLSSPVTLGQPASISCRSSQSLVHSDGNTYLSWLQQRPGQPPRLLIYKISNRFSGVPDRFSGTGAGTDFTLKISRVEAEDVGVYYCMQATQFPHTFGGGTKVEIK

SEQ ID NO: 13 - protein, 108 aa, Homo Sapiens
EIVLTQSPGTLSLSPGERATLSCRASQTVISSYLAWYQQKPGQAPRLLISGASSRATGIPDRFSGSGSGTDFTLTISRLEPEDFAVYYCQQYGSSPRTFGQGTKVEIK

SEQ ID NO: 14 - protein, 106 aa, Homo Sapiens
EIVLTQSPGTLSLSPGERATLSCRASQSVSRLAWYQQKPGQAPRLLIYGASRRATGIPDRFSGSGSGTDFTLTISRLEPEDFAVYYCQQYGSSPRSFGQGTKLEIK

SEQ ID NO: 15 - protein, 107 aa, Homo Sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPPTFGQGTKVEIK

SEQ ID NO: 16 - protein, 113 aa, Homo Sapiens
DIVMTQSPDSLAVSLGERATINCKSSQSVLYSSNNKNYLVWYQQKPGQPPKLFIYWASTRESGVPDRFTGSGSGTDFTLTISSLQAEDVAVYYCQQYYSFPWTFGQGTKVEIK

SEQ ID NO: 17 - protein, 113 aa, Homo Sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTTWTFGQGTKVEIK

SEQ ID NO: 18 - protein, 113 aa, Homo Sapiens
DIVMTQSPDSLAVSLGERATINCKSSQNVLYSSNNKNYLAWYQQKPGQPPKLLIYWASTRESGVPDRFSGSGSGTDFTLTISSLQAEDVAVYFCQQYYGTPRTFGQGTKVEIK

SEQ ID NO: 19 - protein, 113 aa, Homo Sapiens; identical to SEQ ID NO: 18

SEQ ID NO: 20 - protein, 113 aa, Homo Sapiens
DIVMTQSPDSLTVSLGERATINCKSSQSVLYSSNNKNYLAWYQQKPGQPPKLLIYWASTRESGVPDRFGGSGSGTDFTLTISSLQAEDVAVYYCQQYYSISRTFGQGTKVEIK

SEQ ID NO: 21 - protein, 113 aa, Homo Sapiens
DIVMTQSPDSLAVSLGERATINCKSSQSVLYNSNNKNYLAWYQQKPGQPPKLLIYWASTRESGVPDRFSGSGSGTDFTLTISSLQADDVAVYYCQQYYSTTWTFGPGTKVEIK

SEQ ID NO: 22 - protein, 113 aa, Homo Sapiens; identical to SEQ ID NO: 21

SEQ ID NO: 23 - protein, 113 aa, Homo Sapiens
DIVMTQSPDSLAVSLGERATINCKSSQSVLYSSNNKNYLAWYQQKPGQPPKVLIYWASTRKSGVPDRFSGSGSGTDFTLTISGLQAEDVALYYCQQYYSTMFSFGQGTKLEIK

SEQ ID NO: 24 - protein, 113 aa, Homo Sapiens
DIVMTQSPDSLAVSLGERATINCKSSQSVLDSSNNKNYLAWYQQKPGQPPKLLIYWASTRESGVPDRFSGSGSGTDFTLTISSLQAEDVAVFYCHQYYSTPLTFGGGTKVAIK

SEQ ID NO: 25 - protein, 113 aa, Homo Sapiens
DIVMTQSPDSLAVSLGERATINCKSSQSILYRSNNKNYLAWYQQKPGQPPKLLIYWASARESGVPDRFSGSGSGTDFTLTISSLQAEDVAVYFCQQYFITPLTFGGGTKVEIK

SEQ ID NO: 26 - protein, 107 aa, Homo Sapiens
DIQMTQSPSSLSASVGDRVTITCRASQDISHYLAWFQQKPGKAPKSLIYAASSLQSGVPSKFSGSGSGTDFTLTISSLQPEDFATYYCQQYNNYPFTFGPGTKVDIK

SEQ ID NO: 27 - protein, 107 aa, Homo Sapiens
DIQMTQSPSSLSASVGDRVAITCRASQDISNYLAWLQQKPGKAPKSLIYAASSLQSGVPSRFSGSGSGTDFTLTISSLQPEDFATYYCQQYNTYPFTFGPGTKMDIK

SEQ ID NO: 28 - protein, 108 aa, Homo Sapiens
EIVMTQSPATLSVSPGERATLSCRASQSVSSNLAWYQQDPGQAPRLLIYGASRRATGIPARFSGSGSGTEFTLTISSLQSEDFAVYYCQQHNNWPPWTFGQGTKVEIK

SEQ ID NO: 29 - protein, 107 aa, Homo Sapiens
DIQMTQSPSSVSASVGDRVTITCRASQDISRWLAWYQQKPGKAPKLLIYAASSLQSGVPSRFSGSGSGTDFTLTISSLQPEDFATYYCQQANSFPPTFGQGTKVEFK

SEQ ID NO: 30 - protein, 107 aa, Homo Sapiens
DIQMTQSPSSLSASVGDRVTITCRASQSISTYLNWYQQKPGKAPKFLIYAASSLQSGVPSRFSGSGSGTDFTLTISSLQPEDFAAYYCQQSHSAPFTFGPGTKVDIK

SEQ ID NO: 31 - protein, 106 aa, Homo Sapiens
DIQMTQSPSSLSASLGDRVTITCRASQTISIYLNWYQQKPGKAPKLLIYAASSLQSGVPSRFSGSGSGTDFTLTISSLQPEDFATYYCQQSYSTLTFGGGTKVEIK

SEQ ID NO: 32 - protein, 107 aa, Homo Sapiens
DIQMTQSPSSLSASVGDRVTITCRASQSIRSYLNWYQQRPGNAPKLLIYAASSLQSGVPSRVSGSGSGTDFTLTIRSLQPEDFATYYCQQSYSIPLTFGGGTKVEIK

SEQ ID NO: 33 - protein, 107 aa, Homo Sapiens
DIQMTQSPSSRSASVGDRVTITCRASQTISRYLNWYQQKPGKAPKLLIYAASTLQSGVPSRFSGSGSGTDFTLTLSSLQPEDFATYYCQQIYSTSITFGQGTRLEIK

SEQ ID NO: 34 - protein, 107 aa, Homo Sapiens
DIQMTQSPSSLSASVGDRVTITCRASQRISSYLNWYQQKPGKAPKVLIYAESSLQSGVPSRFSGSGSGTDFTLTISSLQPEDFATYYCQQSYITPITFGQGTRLEII

SEQ ID NO: 35 - protein, 107 aa, Homo Sapiens
DIQMTQSPSSLSASVGDRVTITCRASQSISRYLNWYQQKPGKAPKLLIYTASSLQSGVPSRFSGSGSGTDFTLTISSLQPENFATYYCQQSYFTPITFGQGTRLEIK

SEQ ID NO: 36 - protein, 107 aa, Homo Sapiens
DIQMTQSPSSLSASVGDRVTITCRASQSISSYLNWYQQKPGKAPKLLIYTASSLQSGVPSRFSGSGSGTDFTLTFSSLQPEDFATYYCQQSYFSPITFGQGTRLEIK

SEQ ID NO: 37 - protein, 107 aa, Homo Sapiens
DIQMTQSPSSLSASVGDRVTITCRASQSISSYLNWYQQKPGKAPKLLIYTASSLQSGVPSRFSGSGSGTDFTLTLSSLQPEDFASYYCQQSFYTPITFGQGTRLEIK

SEQ ID NO: 38 - protein, 107 aa, Homo Sapiens
DIQMTQSPSSLSASVGDRVTITCRASQSISSYLNWYQQKPGKAPKLLIYTVSSLQSGVPSRFSGSGSGTDFTLTISSLQPEDFATYYCQQSYFTPITFGQGTRLEIK

SEQ ID NO: 39 - protein, 107 aa, Homo Sapiens
DIQMTQSPSSLSASVGDRVTITCRASQSISSYLNWYQQKPGKAPKLLIYTASSLQSGVPSRFSGSGSGTDFTLTISSLQPEDFASYYCQQSFYAPITFGQGTRLEIK

SEQ ID NO: 40 - protein, 107 aa, Homo Sapiens
DIQMTQSPSSLSASVGDRVTITCRASQSISSYLNWYQQKPGKAPKLLIYTASSLQSGVPSRFSGSGSGTDFTLTISSLQPEDFATYYCQQSYFTPITFGQGTRLEIK

SEQ ID NO: 41 - protein, 107 aa, Homo Sapiens
DIQMTQSPSSLSASVGDRVTITCQASQDISNYLNWYQQKPGKAPKLLIYDASNLETGVPSRFSGSGSGTDFTFTISSLQPEDIATYYCQQYDYLPFTFGPGTKVDIK

SEQ ID NO: 42 - protein, 107 aa, Homo Sapiens
DIQMTQSPSSLSASVGDRVTITCQASQDISNSLNWYQQKPGKAPELLIYDASNLETGVPSRFSGSGSGTDFTFTISSLQPEDIATYYCQQCDDLPLTFGGGTKVEIK

SEQ ID NO: 43 - protein, 107 aa, Homo Sapiens
DIQMTQSPSSLSASVGDRVTITCQASQDISDYLNWYQQKPGKAPKLLIYDASNLETGVPSRFSGSGSGTDFTFTISSLQPEDIATYYCQHYDNLPLTFGGGTKVEIK

SEQ ID NO: 44 - protein, 107 aa, Homo Sapiens
DIQMTQSPSSLSASVGDRVAITCQASQDISNYLNWYQQKPGKAPKLLIYDASNLETGVPSRFSGSGSGTDFTFTISSLQPEDIATYYCQQYDNLPLTFGGGTKVEIK

SEQ ID NO: 45 - protein, 107 aa, Homo Sapiens
DIQMTQSPSSLSASVGDRVTITCQASQDISNSLNWYQQKPGKAPKLLIYDASILETGVPSRFSGSGSETDFTFTISSLQPEDIATYYCQQCDILPLSFGGGTKVEIK

SEQ ID NO: 46 - protein, 107 aa, Homo Sapiens
DIQMTQSPSSLSASVGDRVTITCQASQDISNSLNWYQQKPGKAPKLLIYDASNLETGVPSRFSGSGSGTDFTFTISSLQPEDIATYYCQQYDNLPLAFGGGTKVEIR

SEQ ID NO: 47 - protein, 107 aa, Homo Sapiens
DIQMTQSPSSLSASVGDGVTITCQASQDITNYLNWYQQKPGKAPKLLIYDASNLETGVPSRFSGSGSGTDFTFTISSLQPEDIATYYCQQYDSLPITFGQGTRLEIK

SEQ ID NO: 48 - protein, 107 aa, Homo Sapiens
DIQMTQSPSSLSASVGDRVTITCQASQDISNYLNWYQQKLGKAPKLLIHDASNLETGVPSRFSGSGSGTDFTFTISSLQPEDIATYYCQQYDNLPITFGQGTRLEIK

SEQ ID NO: 49 - protein, 107 aa, Homo Sapiens
DIQMTQSPSSLSASVGDRVTITCQASQDISDYLNWYQQKPGKAPKLLIYDASNLETGVPSRFSGSGSGTDFTFTISSLQPEDIATYYCQHYDNLPITFGQGTRLEIK

SEQ ID NO: 50 - protein, 107 aa, Homo Sapiens
DIQMTQSPSSLSASVGDRVTITCQASQDISNSLNWYQQKPGKAPKLLIYDASNLETGVPSRFSGSGSGTDFTFTISSLQPEDIATYYCQHYDNLPITFGQGTRLEIK

SEQ ID NO: 51 - protein, 120 aa, Homo Sapiens
QVQLVQSGAEVKKPGASVKVSCKASGYTFTSYGISWVRQAPGQGLEWMGWISASNGNTNYAQKLQDRVTMTTDTSTSTAYMELRSLRSDDTAVYYCAREDNWNYGFFDYWGQGTLVTVSS

SEQ ID NO: 52 - protein, 117 aa, Homo Sapiens
QVHLVQSGAEVKKPGASVKVSCKVSGYTFTGHYMHWVRQAPGQGLEWMGWINPNSGGTNCAQKFQGRVTMTRDTSISTAYMELSRLRSDDTAVYYCARSIAVALDYWGQGTLVTVSS

SEQ ID NO: 53 - protein, 117 aa, Homo Sapiens
QVQLVQSGAEVKKPGASVKVSCKASGYTFTGYYMHWVRQAPGQGLEWMGWINPNSGGTNHTQKFQGRVTMTRDTSISTAYMELSRLRSDDTAVYYCARSIAVALDYWGQGTLVTVSS

SEQ ID NO: 54 - protein, 122 aa, Homo Sapiens
QVQLVQSGAEVRKPGASVKVSCKVSGYTLTELSMHWVRQAPGKGLEWMGSFDPEDGETIYAQKFQGRVTMLEDTSTDTAYMELSSLRSEDTAVYYCATEGDGGYYYYGMDVWGQGTTVTVSS

SEQ ID NO: 55 - protein, 122 aa, Homo Sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARMYSSGWYGVFDYWGQGTLVTVSS

SEQ ID NO: 56 - protein, 123 aa, Homo Sapiens
QVTLKESGPVLVKPTETLTLTCTVSGFSLSNARMGVSWIRQPPGKALEWLVLIFSNDEKSYSTSLKSRLTISKDTSKSQVVLTMTNMDPVDTATYYCARVYSSGWSFYGMDVWGQGTTVTVSS

SEQ ID NO: 57 - protein, 118 aa, Homo Sapiens
QITLKESGPTLVKPTQTLTLTCTFSGFSLSTGGVGVGWIRQPPGKALEWLALIYWNDDKRYSPSLKSRLTITKDTSKNQVVLTMTNMDPVDTATYYCAHRRELPFDYWGQGTLVTVSS

SEQ ID NO: 58 - protein, 118 aa, Homo Sapiens
QITLKESGPTLVKPTQTLTLTCTFSGFSLSTGGVGVGWIRQPPGKALEWLALIYWNDDKRYSPSLKSRLTITKDTSKTQVVLTVTDMDPVDTATYYCAHRNWTPFDYWGQGTLVTVSS

SEQ ID NO: 59 - protein, 118 aa, Homo Sapiens
QITLKESGPTLVKPTQTLTLTCTFSGFSLNTGGVGVGWIRQPPGKALEWLALIYWNDDKRYSPSLKSRLTITKDTSKNQVVLTMTNMDPVDTATYYCAHRLELPFDYWGQGTLVTVSS

SEQ ID NO: 60 - protein, 118 aa, Homo Sapiens
QITLKESGPTLVKPTQTLTLTCTFSGFSLSTGGVGVGWIRQPPGKALEWLALIYWNDDKRYSPSLKSRLTITKDTSKNQVVLTMTNLDPVDTATYYCAHRREVPFDYWGQGTLVTVSS

SEQ ID NO: 61 - protein, 118 aa, Homo Sapiens
QITLKESGPTLVKPTQTLTLTCTFSGFSLSTGGVGVGWIRQPPGKALEWLALIYWNVEKRYSPSLRSRLTITKATSKNQVVLTMTNMDPVDTATYYCAHRHTNPFEYWGQGTLVTVSS

SEQ ID NO: 62 - protein, 118 aa, Homo Sapiens
QITLKESGPTLVKPTQTLTLTCTFSGFSLSTGGVGVGWIRQPPGKALEWLALIYWNDDKRYSPSLKSRLTITKDTSKNQVVLTMTNMDPVDTATYYCAHRGELPFDYWGQGTLVTVSS

SEQ ID NO: 63 - protein, 121 aa, Homo Sapiens
EVQLVESGGGLVKPGGSLRLSCAASGFPFSRYSMNWVRQAPGKGLEWVSAISSSSSYIYYADSVKGRFTISRDNAKNSLYLQMNSLRAEDTAVYYCARDRVGATPDAFDIWGQGTMVTVSS

SEQ ID NO: 64 - protein, 128 aa, Homo Sapiens
EVQLLESGGGLVQPGGSLRLSCAASGFTFSSYAMNWVRQAPGKGLEWVSAISGSGGSTYYADSVKGRFTISRDNSKNTLYLQMNSLRAEDTAVYYCAKEGIAVAGTAEYYYYYAMDVWGQGTTVTVSS

SEQ ID NO: 65 - protein, 125 aa, Homo Sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEGIAARDSYYYYAMDVWGQGTTVTVSS

SEQ ID NO: 66 - protein, 125 aa, Homo Sapiens
EVQLLESGGGLVQPGGSLRLSCTASGFTFSSYAMSWVRQAPGKGLEWVSAISGSGGSTYYADSVKGRFTISRDNSKNTLYLQMNSLRAEDTAEYYCAKEGIAGRDSYYYYGMDVWGQGTTVTVSS

SEQ ID NO: 67 - protein, 123 aa, Homo Sapiens
QVQLVESGGGVVQPGRSLRLSCAASGFTFSSYGMHWVRQAPGKGLEWVAFISDDGSTKYYADSVKGRFTISRDNSMNTLYLQMNSLRAEDTAVYYCARSYYDSSGYYYGFDYWGQGTLVTVSS

SEQ ID NO: 68 - protein, 114 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARNVIDYWGQGTLVTVSS

SEQ ID NO: 69 - protein, 120 aa, Homo Sapiens
QVQLVESGGGVVQPGRSLRLSCAASGFTFSSYDMHWVRQAPGKGLEWVAVIWYDGSIKYYADSVKGRFTISRDNSKNTLYLQMNSLRAEDTAVYYCARGGATGAEYFQHWGQGTLVTVSS

SEQ ID NO: 70 - protein, 118 aa, Homo Sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCVLLWFGETFDYWGQGSLVTVSP

SEQ ID NO: 71 - protein, 115 aa, Homo Sapiens
QVQLVESGGGVVQPGRSLRLSCAASGFTFSSYGMHWVRQAPGKGLEWVAVIWSDGSNKYYADSVKGRFTISRDNSKNTLYLQMNSLRAEDTAVYYCARNLPFDYWGQGTLVTVSS

SEQ ID NO: 72 - protein, 123 aa, Homo Sapiens
QVQLVESGGGVVQPGRSLRLSCAASGFTFSSYGMHWVRQAPGKGLEWVAVIWDDGSNQYYTDSVKGRFTVSRDNSKNTLFLQMNSLRAEDTAVYYCARSHYGGDYDYYGMDVWGQGTTVTVSS

SEQ ID NO: 73 - protein, 120 aa, Homo Sapiens
QVQLVESGGGVVQPGRSLRLSCAASGFTFSSYGMHWVRQAPGKGLEWVAVIWYDGSNKRYVDSVKGRFTISRDNSKNTLYLQMNSLRAEDTAVYYCARDGWQQQAPFDYWGQGTLVTVSS

SEQ ID NO: 74 - protein, 118 aa, Homo Sapiens
QVQLVESGGGVVQPGRSLRLSCAASGFTFRSHGMHWVRQAPGKGLEWVAVIWYDGSNKNYADSVRGRFTISRDNSKNTLDLQMNSLRAEDTAVYYCARWGISAPFDCWGQGTLVTVSS

SEQ ID NO: 75 - protein, 115 aa, Homo Sapiens
EVQLVESGGGLVQPGGSLRLSCAASGFTFSAYSMNWVRQAPGKGLEWVSYISSSGRTIYYADSVKGRFTISRDNAKNSLFLQMNSLRDEDTAVYYCALWAPFDYWGQGTLVTVSS

SEQ ID NO: 76 - protein, 125 aa, Homo Sapiens
EVQLVESGGGLVQPGGSLRLSCAASGFTFSSYSMNWVRQAPGKGLEWVSHISSSSRTIYYADSVKGRFTISRDNAKNSVYLQMNSLRDEDTAVYYCARDGYNWNGGGNYYGMDVWGQGTTVTVSS

SEQ ID NO: 77 - protein, 125 aa, Homo Sapiens
EVQLVESGGGLVQPGGSLRLSCAASGFTFSSYSMNWVRQAPGKGLEWVSHISRSSRTIYYADSVKGRFTISRDNAKNSLYLQMNSLRDEDTAVYYCARDGYNWNNGGYYYGMDVWGQGTTVTVSS

SEQ ID NO: 78 - protein, 118 aa, Homo Sapiens
QVQLQESGPGLVKPSQTLSLTCTVSGGSVSSGGYYWSWIRQHPGKGLEWIGYIHSSGSTYYNPSLKSRVTISVDTSKNQFSLNLSSVTAADTAVYYCARGPYYGMDVWGQGTTVTVSS

SEQ ID NO: 79 - protein, 127 aa, Homo Sapiens
QVQLQESGPGLVKPSQTLSLTCTVSGGSISRGGYYWSWIRQHPGKGLEWIGYIYHSGSTYYNPSLKSRVNMSVDTSKNQFSLKLSSVTAADTAVYYCARALRGIVLMVYVLGALDIWGQGTKVTVSS

SEQ ID NO: 80 - protein, 127 aa, Homo Sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDETVVRGLIRYCYGMDVWGQGTTVTVSS

SEQ ID NO: 81 - protein, 122 aa, Homo Sapiens
QVQLQESGPGLVKPSQTLSLNCTVSGGSISSGGYYWSWIRQHPGKGLEWIGYIHYSGSTYYNPSLKSRITISADTSKNQFSLKLNSVTAADTAVYYCARDRGGGDYGRMDVWGQGTTVTVSS

SEQ ID NO: 82 - protein, 122 aa, Homo Sapiens; identical to SEQ ID NO: 81

SEQ ID NO: 83 - protein, 119 aa, Homo Sapiens
QVQLQESGPGLVKPSQTLSLTCTVSGGSISSGGYYWSWIRQHPGKGLEWIGYIHSSGSTYYNPSLKSRITKSVDTSKNQFSLKLSSVTAADTAVYYCARSNNYGCFALWGRGTLVTVSS

SEQ ID NO: 84 - protein, 119 aa, Homo Sapiens; identical to SEQ ID NO: 83

SEQ ID NO: 85 - protein, 133 aa, Homo Sapiens
QVQLQESGPGLVKPSQTLSLTCTVSGGSISSGGYYWSWIRQHPGKGLEWIGYIHYSGSTYYNPSLKSRVTISVDTSKNQFSLKLSSVTAADTAVYYCASGYNYGLYYYDSSGYPSYYYGMDVWGQGTTVTVSS

SEQ ID NO: 86 - protein, 125 aa, Homo Sapiens
QVQLQESGPGLVKPSQTLSLTCTVSGGSISSGDYYWNWVRQHPGKGLEWIGYIYYSGGTYYNPSLKSRVTISVDTSKNQFSLKLFSVTAADTAVYFCARTYYDILTGYPFYFDYWGQGTLVTVSS

SEQ ID NO: 87 - protein, 125 aa, Homo Sapiens; identical to SEQ ID NO: 86

SEQ ID NO: 88 - protein, 120 aa, Homo Sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGYSSSWYWFDPWGQGTLVTVSS

SEQ ID NO: 89 - protein, 120 aa, Homo Sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGGYSSSWFWFDPWGQGTLVTVSS

SEQ ID NO: 90 - protein, 123 aa, Homo Sapiens
QVQLQESGPGLVKPSETLSLTCTVSGGSISSYYWSWIRQPAGKGLEWIGRIYTSGTTNYNPSLKSRVTMSVDTSKNQFSLKLSSVTAADTAVYYCARDGYSYGHYYYYGMDVWGQGTTVTVSS

SEQ ID NO: 91 - protein, 126 aa, Homo Sapiens
QVQLQESGPGLVKPSETLSLTCTVSGGSVSSGGSYWSWIRQPPGKGLEWIGYIYYSGSTNYNPSLKSRVTISIVTSRNQFSLKLSSVTAADTAVYYCARSALRYFDWLFSDVSDIWGQGTMVTVSS

SEQ ID NO: 92 - protein, 120 aa, Homo Sapiens
EVQLVQSGAELKKPGESLKISCKGSGYRFTSYWIGWVRQMPGKGLEWMGIIYPDDSDTRYSPSFQGQVTISADKSISTAYLQWSSLKASDTAMYYCARQKSYGYSYFDYWGQGTLVTVSS

SEQ ID NO: 93 - protein, 121 aa, Homo Sapiens
EVQLVQSGAEVKKPGESLKISCKGSGYSFTSYWIGWVRQMPGKGLEWMGIIYPDDSDARYSPSFQGQVTISADKSINTAYLQWSSLKASDTAMYYCARQGYGSGWGYFDYWGQGTLVTVSS

SEQ ID NO: 94 - protein, 127 aa, Homo Sapiens
EVQLVQSGAEVKKPGESLKISCKGSGYSFTSYWIGWVRQMPGKGLEWMGIIYPGDSDIRYSPSFQGQVTISADKSISTAYLQWSSLKASDTAMYYCARQGLAVAGTSYYYYYGMDVWGQGTTVTVSS

SEQ ID NO: 95 - protein, 125 aa, Homo Sapiens
QVQLQQSGPGLVKPSQTLSLTCAISGDSVSSYSAAWNWIRQSPSRGLEWLGRTYCRSKWYNDYAVSVKSRITINPDTSKNQFSLQLNSVTPEDTAVYYCARDRAVAGYYYGMDVWGQGTTVTVSS

SEQ ID NO: 96 - protein, 16 aa, Homo Sapiens
RSSQSLVYSDGNTYLN

SEQ ID NO: 97 - protein, 16 aa, Homo Sapiens
KSSQSLLHSDGKTYLY

SEQ ID NO: 98 - protein, 11 aa, Homo Sapiens
RASQGIANYLA

SEQ ID NO: 99 - protein, 11 aa, Homo Sapiens
RASQGISNDLA

SEQ ID NO: 100 - protein, 16 aa, Homo Sapiens
RSSQSLVHSDGNTYLS

SEQ ID NO: 101 - protein, 12 aa, Homo Sapiens
RASQTVISSYLA

SEQ ID NO: 102 - protein, 10 aa, Homo Sapiens
RASQSVSRLA

SEQ ID NO: 103 - protein, 11 aa, Homo Sapiens
RASQGIRNDLG

SEQ ID NO: 104 - protein, 17 aa, Homo Sapiens
KSSQSVLYSSNNKNYLV

SEQ ID NO: 105 - protein, 17 aa, Homo Sapiens
KSSQSVLYSSNNKNYLA

SEQ ID NO: 106 - protein, 17 aa, Homo Sapiens
KSSQNVLYSSNNKNYLA

SEQ ID NO: 107 - protein, 17 aa, Homo Sapiens
KSSQSVLYNSNNKNYLA

SEQ ID NO: 108 - protein, 17 aa, Homo Sapiens
KSSQSVLDSSNNKNYLA

SEQ ID NO: 109 - protein, 17 aa, Homo Sapiens
KSSQSILYRSNNKNYLA

SEQ ID NO: 110 - protein, 11 aa, Homo Sapiens
RASQDISHYLA

SEQ ID NO: 111 - protein, 11 aa, Homo Sapiens
RASQDISNYLA

SEQ ID NO: 112 - protein, 11 aa, Homo Sapiens
RASQSVSSNLA

SEQ ID NO: 113 - protein, 11 aa, Homo Sapiens
RASQDISRWLA

SEQ ID NO: 114 - protein, 11 aa, Homo Sapiens
RASQSISTYLN

SEQ ID NO: 115 - protein, 11 aa, Homo Sapiens
RASQTISIYLN

SEQ ID NO: 116 - protein, 11 aa, Homo Sapiens
RASQSIRSYLN

SEQ ID NO: 117 - protein, 11 aa, Homo Sapiens
RASQTISRYLN

SEQ ID NO: 118 - protein, 11 aa, Homo Sapiens
RASQRISSYLN

SEQ ID NO: 119 - protein, 11 aa, Homo Sapiens
RASQSISRYLN

SEQ ID NO: 120 - protein, 11 aa, Homo Sapiens
RASQSISSYLN

SEQ ID NO: 121 - protein, 11 aa, Homo Sapiens
QASQDISNYLN

SEQ ID NO: 122 - protein, 11 aa, Homo Sapiens
QASQDISNSLN

SEQ ID NO: 123 - protein, 11 aa, Homo Sapiens
QASQDISDYLN

SEQ ID NO: 124 - protein, 11 aa, Homo Sapiens
QASQDITNYLN

SEQ ID NO: 125 - protein, 7 aa, Homo Sapiens
KVSNWDS

SEQ ID NO: 126 - protein, 7 aa, Homo Sapiens
EVSNRFS

SEQ ID NO: 127 - protein, 7 aa, Homo Sapiens
VASTLQS

SEQ ID NO: 128 - protein, 7 aa, Homo Sapiens
AASTLQS

SEQ ID NO: 129 - protein, 7 aa, Homo Sapiens
KISNRFS

SEQ ID NO: 130 - protein, 7 aa, Homo Sapiens
GASSRAT

SEQ ID NO: 131 - protein, 7 aa, Homo Sapiens
GASRRAT

SEQ ID NO: 132 - protein, 7 aa, Homo Sapiens
AASSLQS

SEQ ID NO: 133 - protein, 7 aa, Homo Sapiens
WASTRES

SEQ ID NO: 134 - protein, 7 aa, Homo Sapiens
WASTRKS

SEQ ID NO: 135 - protein, 7 aa, Homo Sapiens
WASARES

SEQ ID NO: 136 - protein, 7 aa, Homo Sapiens
AESSLQS

SEQ ID NO: 137 - protein, 7 aa, Homo Sapiens
TASSLQS

SEQ ID NO: 138 - protein, 7 aa, Homo Sapiens
TVSSLQS

SEQ ID NO: 139 - protein, 7 aa, Homo Sapiens
DASNLET

SEQ ID NO: 140 - protein, 7 aa, Homo Sapiens
DASILET

SEQ ID NO: 141 - protein, 9 aa, Homo Sapiens
MQSTHWPIT

SEQ ID NO: 142 - protein, 9 aa, Homo Sapiens
IQGTHWPTT

SEQ ID NO: 143 - protein, 9 aa, Homo Sapiens
MQGTHWPIT

SEQ ID NO: 144 - protein, 9 aa, Homo Sapiens
MQGIQLPCS

SEQ ID NO: 145 - protein, 9 aa, Homo Sapiens
MQSIQLPLT

SEQ ID NO: 146 - protein, 9 aa, Homo Sapiens
MQSIQLPIT

SEQ ID NO: 147 - protein, 9 aa, Homo Sapiens
QNYNSAPFT

SEQ ID NO: 148 - protein, 9 aa, Homo Sapiens
QKYNSVPLT

SEQ ID NO: 149 - protein, 9 aa, Homo Sapiens
MQATQFPHT

SEQ ID NO: 150 - protein, 9 aa, Homo Sapiens
QQYGSSPRT

SEQ ID NO: 151 - protein, 9 aa, Homo Sapiens
QQYGSSPRS

SEQ ID NO: 152 - protein, 9 aa, Homo Sapiens
LQHNSYPPT

SEQ ID NO: 153 - protein, 9 aa, Homo Sapiens
QQYYSFPWT

SEQ ID NO: 154 - protein, 9 aa, Homo Sapiens
QQYYSTTWT

SEQ ID NO: 155 - protein, 9 aa, Homo Sapiens
QQYYGTPRT

SEQ ID NO: 156 - protein, 9 aa, Homo Sapiens
QQYYSISRT

SEQ ID NO: 157 - protein, 9 aa, Homo Sapiens
QQYYSTMFS

SEQ ID NO: 158 - protein, 9 aa, Homo Sapiens
HQYYSTPLT

SEQ ID NO: 159 - protein, 9 aa, Homo Sapiens
QQYFITPLT

SEQ ID NO: 160 - protein, 9 aa, Homo Sapiens
QQYNNYPFT

SEQ ID NO: 161 - protein, 9 aa, Homo Sapiens
QQYNTYPFT

SEQ ID NO: 162 - protein, 10 aa, Homo Sapiens
QQHNNWPPWT

SEQ ID NO: 163 - protein, 9 aa, Homo Sapiens
QQANSFPPT

SEQ ID NO: 164 - protein, 9 aa, Homo Sapiens
QQSHSAPFT

SEQ ID NO: 165 - protein, 8 aa, Homo Sapiens
QQSYSTLT

SEQ ID NO: 166 - protein, 9 aa, Homo Sapiens
QQSYSIPLT

SEQ ID NO: 167 - protein, 9 aa, Homo Sapiens
QQIYSTSIT

SEQ ID NO: 168 - protein, 9 aa, Homo Sapiens
QQSYITPIT

SEQ ID NO: 169 - protein, 9 aa, Homo Sapiens
QQSYFTPIT

SEQ ID NO: 170 - protein, 9 aa, Homo Sapiens
QQSYFSPIT

SEQ ID NO: 171 - protein, 9 aa, Homo Sapiens
QQSFYTPIT

SEQ ID NO: 172 - protein, 9 aa, Homo Sapiens
QQSFYAPIT

SEQ ID NO: 173 - protein, 9 aa, Homo Sapiens
QQYDYLPFT

SEQ ID NO: 174 - protein, 9 aa, Homo Sapiens
QQCDDLPLT

SEQ ID NO: 175 - protein, 9 aa, Homo Sapiens
QHYDNLPLT

SEQ ID NO: 176 - protein, 9 aa, Homo Sapiens
QQYDNLPLT

SEQ ID NO: 177 - protein, 9 aa, Homo Sapiens
QQCDILPLS

SEQ ID NO: 178 - protein, 9 aa, Homo Sapiens
QQYDNLPLA

SEQ ID NO: 179 - protein, 9 aa, Homo Sapiens
QQYDSLPIT

SEQ ID NO: 180 - protein, 9 aa, Homo Sapiens
QQYDNLPIT

SEQ ID NO: 181 - protein, 9 aa, Homo Sapiens
QHYDNLPIT

SEQ ID NO: 182 - protein, 10 aa, Homo Sapiens
GYTFTSYGIS

SEQ ID NO: 183 - protein, 10 aa, Homo Sapiens
GYTFTGHYMH

SEQ ID NO: 184 - protein, 10 aa, Homo Sapiens
GYTFTGYYMH

SEQ ID NO: 185 - protein, 10 aa, Homo Sapiens
GYTLTELSMH

SEQ ID NO: 186 - protein, 12 aa, Homo Sapiens
GFSLSNARMGVS

SEQ ID NO: 187 - protein, 12 aa, Homo Sapiens
GFSLSTGGVGVG

SEQ ID NO: 188 - protein, 12 aa, Homo Sapiens
GFSLNTGGVGVG

SEQ ID NO: 189 - protein, 10 aa, Homo Sapiens
GFPFSRYSMN

SEQ ID NO: 190 - protein, 10 aa, Homo Sapiens
GFTFSSYAMN

SEQ ID NO: 191 - protein, 10 aa, Homo Sapiens
GFTFSSYAMS

SEQ ID NO: 192 - protein, 10 aa, Homo Sapiens
GFTFSSYGMH

SEQ ID NO: 193 - protein, 10 aa, Homo Sapiens
GFTFSSYDMH

SEQ ID NO: 194 - protein, 10 aa, Homo Sapiens
GFTFRSHGMH

SEQ ID NO: 195 - protein, 10 aa, Homo Sapiens
GFTFSAYSMN

SEQ ID NO: 196 - protein, 10 aa, Homo Sapiens
GFTFSSYSMN

SEQ ID NO: 197 - protein, 12 aa, Homo Sapiens
GGSVSSGGYYWS

SEQ ID NO: 198 - protein, 12 aa, Homo Sapiens
GGSISRGGYYWS

SEQ ID NO: 199 - protein, 12 aa, Homo Sapiens
GGSISSGGYYWS

SEQ ID NO: 200 - protein, 12 aa, Homo Sapiens
GGSISSGDYYWN

SEQ ID NO: 201 - protein, 10 aa, Homo Sapiens
GGSFSGYYWS

SEQ ID NO: 202 - protein, 10 aa, Homo Sapiens
GGSISSYYWS

SEQ ID NO: 203 - protein, 12 aa, Homo Sapiens
GGSVSSGGSYWS

SEQ ID NO: 204 - protein, 10 aa, Homo Sapiens
GYRFTSYWIG

SEQ ID NO: 205 - protein, 10 aa, Homo Sapiens
GYSFTSYWIG

SEQ ID NO: 206 - protein, 12 aa, Homo Sapiens
GDSVSSYSAAWN

SEQ ID NO: 207 - protein, 17 aa, Homo Sapiens
WISASNGNTNYAQKLQD

SEQ ID NO: 208 - protein, 17 aa, Homo Sapiens
WINPNSGGTNCAQKFQG

SEQ ID NO: 209 - protein, 17 aa, Homo Sapiens
WINPNSGGTNHTQKFQG

SEQ ID NO: 210 - protein, 17 aa, Homo Sapiens
SFDPEDGETIYAQKFQG

SEQ ID NO: 211 - protein, 16 aa, Homo Sapiens
HIFSNDEKSYSTSLKS

SEQ ID NO: 212 - protein, 16 aa, Homo Sapiens
LIFSNDEKSYSTSLKS

SEQ ID NO: 213 - protein, 16 aa, Homo Sapiens
LIYWNDDKRYSPSLKS

SEQ ID NO: 214 - protein, 16 aa, Homo Sapiens
LIYWNVEKRYSPSLRS

SEQ ID NO: 215 - protein, 17 aa, Homo Sapiens
AISSSSSYIYYADSVKG

SEQ ID NO: 216 - protein, 17 aa, Homo Sapiens
AISGSGGSTYYADSVKG

SEQ ID NO: 217 - protein, 17 aa, Homo Sapiens
FISDDGSTKYYADSVKG

SEQ ID NO: 218 - protein, 17 aa, Homo Sapiens
VIWYDGSNKYYADSVKG

SEQ ID NO: 219 - protein, 17 aa, Homo Sapiens
VIWYDGSIKYYADSVKG

SEQ ID NO: 220 - protein, 17 aa, Homo Sapiens
VIWSDGSNKYYADSVKG

SEQ ID NO: 221 - protein, 17 aa, Homo Sapiens
VIWDDGSNQYYTDSVKG

SEQ ID NO: 222 - protein, 17 aa, Homo Sapiens
VIWYDGSNKRYVDSVKG

SEQ ID NO: 223 - protein, 17 aa, Homo Sapiens
VIWYDGSNKNYADSVRG

SEQ ID NO: 224 - protein, 17 aa, Homo Sapiens
YISSSGRTIYYADSVKG

SEQ ID NO: 225 - protein, 17 aa, Homo Sapiens
HISSSSRTIYYADSVKG

SEQ ID NO: 226 - protein, 17 aa, Homo Sapiens
HISRSSRTIYYADSVKG

SEQ ID NO: 227 - protein, 16 aa, Homo Sapiens
YIHSSGSTYYNPSLKS

SEQ ID NO: 228 - protein, 16 aa, Homo Sapiens
YIYHSGSTYYNPSLKS

SEQ ID NO: 229 - protein, 16 aa, Homo Sapiens
YIYYSGSTYYNPSLKS

SEQ ID NO: 230 - protein, 16 aa, Homo Sapiens
YIHYSGSTYYNPSLKS

SEQ ID NO: 231 - protein, 16 aa, Homo Sapiens
YIYYSGGTYYNPSLKS

SEQ ID NO: 232 - protein, 16 aa, Homo Sapiens
EINHSGSTNYNPSLKS

SEQ ID NO: 233 - protein, 16 aa, Homo Sapiens
RIYTSGTTNYNPSLKS

SEQ ID NO: 234 - protein, 16 aa, Homo Sapiens
YIYYSGSTNYNPSLKS

SEQ ID NO: 235 - protein, 17 aa, Homo Sapiens
IIYPDDSDTRYSPSFQG

SEQ ID NO: 236 - protein, 17 aa, Homo Sapiens
IIYPDDSDARYSPSFQG

SEQ ID NO: 237 - protein, 17 aa, Homo Sapiens
IIYPGDSDIRYSPSFQG

SEQ ID NO: 238 - protein, 18 aa, Homo Sapiens
RTYCRSKWYNDYAVSVKS

SEQ ID NO: 239 - protein, 11 aa, Homo Sapiens
EDNWNYGFFDY

SEQ ID NO: 240 - protein, 8 aa, Homo Sapiens
SIAVALDY

SEQ ID NO: 241 - protein, 13 aa, Homo Sapiens
EGDGGYYYYGMDV

SEQ ID NO: 242 - protein, 12 aa, Homo Sapiens
MYSSGWYGVFDY

SEQ ID NO: 243 - protein, 13 aa, Homo Sapiens
VYSSGWSFYGMDV

SEQ ID NO: 244 - protein, 8 aa, Homo Sapiens
RRELPFDY

SEQ ID NO: 245 - protein, 8 aa, Homo Sapiens
RNWTPFDY

SEQ ID NO: 246 - protein, 8 aa, Homo Sapiens
RLELPFDY

SEQ ID NO: 247 - protein, 8 aa, Homo Sapiens
RREVPFDY

SEQ ID NO: 248 - protein, 8 aa, Homo Sapiens
RHTNPFEY

SEQ ID NO: 249 - protein, 8 aa, Homo Sapiens
RGELPFDY

SEQ ID NO: 250 - protein, 12 aa, Homo Sapiens
DRVGATPDAFDI

SEQ ID NO: 251 - protein, 19 aa, Homo Sapiens
EGIAVAGTAEYYYYYAMDV

SEQ ID NO: 252 - protein, 16 aa, Homo Sapiens
EGIAARDSYYYYAMDV

SEQ ID NO: 253 - protein, 16 aa, Homo Sapiens
EGIAGRDSYYYYGMDV

SEQ ID NO: 254 - protein, 14 aa, Homo Sapiens
SYYDSSGYYYGFDY

SEQ ID NO: 255 - protein, 5 aa, Homo Sapiens
NVIDY

SEQ ID NO: 256 - protein, 11 aa, Homo Sapiens
GGATGAEYFQH

SEQ ID NO: 257 - protein, 9 aa, Homo Sapiens
LWFGETFDY

SEQ ID NO: 258 - protein, 6 aa, Homo Sapiens
NLPFDY

SEQ ID NO: 259 - protein, 14 aa, Homo Sapiens
SHYGGDYDYYGMDV

SEQ ID NO: 260 - protein, 11 aa, Homo Sapiens
DGWQQQAPFDY

SEQ ID NO: 261 - protein, 9 aa, Homo Sapiens
WGISAPFDC

SEQ ID NO: 262 - protein, 6 aa, Homo Sapiens
WAPFDY

SEQ ID NO: 263 - protein, 16 aa, Homo Sapiens
DGYNWNGGGNYYGMDV

SEQ ID NO: 264 - protein, 16 aa, Homo Sapiens
DGYNWNNGGYYYGMDV

SEQ ID NO: 265 - protein, 8 aa, Homo Sapiens
GPYYGMDV

SEQ ID NO: 266 - protein, 17 aa, Homo Sapiens
ALRGIVLMVYVLGALDI

SEQ ID NO: 267 - protein, 17 aa, Homo Sapiens
DETVVRGLIRYCYGMDV

SEQ ID NO: 268 - protein, 12 aa, Homo Sapiens
DRGGGDYGRMDV

SEQ ID NO: 269 - protein, 9 aa, Homo Sapiens
SNNYGCFAL

SEQ ID NO: 270 - protein, 23 aa, Homo Sapiens
GYNYGLYYYDSSGYPSYYYGMDV

SEQ ID NO: 271 - protein, 15 aa, Homo Sapiens
TYYDILTGYPFYFDY

SEQ ID NO: 272 - protein, 12 aa, Homo Sapiens
GGYSSSWYWFDP

SEQ ID NO: 273 - protein, 12 aa, Homo Sapiens
GGYSSSWFWFDP

SEQ ID NO: 274 - protein, 15 aa, Homo Sapiens
DGYSYGHYYYYGMDV

SEQ ID NO: 275 - protein, 16 aa, Homo Sapiens
SALRYFDWLFSDVSDI

SEQ ID NO: 276 - protein, 11 aa, Homo Sapiens
QKSYGYSYFDY

SEQ ID NO: 277 - protein, 12 aa, Homo Sapiens
QGYGSGWGYFDY

SEQ ID NO: 278 - protein, 18 aa, Homo Sapiens
QGLAVAGTSYYYYYGMDV

SEQ ID NO: 279 - protein, 13 aa, Homo Sapiens
DRAVAGYYYGMDV

(19) United States
(12) Patent Application Publication
(10) Pub. No.: US 2015/0037336 A1
(43) Pub. Date: Feb. 5, 2015

(54) COMBINATION OF HB-EGF BINDING PROTEIN AND EGFR INHIBITOR

(71) Applicant: U3 PHARMA GMBH, Martinsried (DE)

(72) Inventor: Esther Zwick Wallasch, Martinsried (DE)

(73) Assignee: U3 PHARMA GMBH, Martinsried (DE)

(21) Appl. No.: 14/378,781

(22) PCT Filed: Feb. 20, 2013
(86) PCT No.: PCT/EP2013/053347

§ 371 Date: Aug. 14, 2014

Related U.S. Application Data
(60) Provisional application No. 61/601,652, filed on Feb. 22, 2012.

(51) Int. Cl. A61K 39/395; A61K 31/517

(52) U.S. Cl. CPC A61K 39/3955; A61K 31/517; A61K 2039/505
USPC 424/135.1; 424/158.1; 424/133.1; 424/136.1; 424/141.1; 424/142.1

(57) ABSTRACT

The present application relates to the combined use of an antigen-binding protein that binds HB-EGF and an EGFR tyrosine kinase inhibitor in medical treatment.

The present application relates to the combined use of an antigen-binding protein that binds HB-EGF and an EGFR tyrosine kinase inhibitor in medical treatment.

### BACKGROUND

The human epidermal growth factor receptor (HER) family comprises four distinct receptor tyrosine kinases referred to as HER1 (or erbB1), HER2 (or erbB2), HER3 (or erbB3), and HER4 (or erbB4). HER1 is also commonly referred to as epidermal growth factor receptor (EGFR). With the exception of HER3, these receptors have phospho-acceptor target specific intrinsic protein tyrosine kinase activities. Members of the HER family are expressed in most epithelial cells as well as in a number of different tumor cell types. For example, receptors of the HER family are expressed in tumor cells of epithelial origin, and of mesenchymal origin. Moreover, HER receptor tyrosine kinases are involved in cell proliferation and angiogenesis, which are associated with diseases such as cancer. For example, EGFR is frequently over-expressed or aberrantly activated in breast cancers, liver cancers, kidney cancers, leukemia, bronchial cancers, pancreatic cancers and gastrointestinal cancers such as colon, rectal or stomach cancers. High levels of the EGF receptor also correlate with poor prognosis and response to treatment (Wright et al., 1992, Br. J. Cancer 65:118-121). Thus, disruption of signal transduction from and to these kinases would have an anti-proliferative, and as such, therapeutic effect upon a number of cancer and tumor cell types.

The enzymatic activity of receptor tyrosine kinases can be stimulated by over-expression and/or by ligand-mediated dimerization (Heldin, 1995, Cell 80:213-223). Activation of receptor homodimers and heterodimers results in phosphorylation of tyrosine residues on the receptors, which in turn phosphorylate tyrosine residues of other molecules, including intracellular proteins. (Ullrich et al., 1990, Cell 61:203-212). This is followed by the activation of intracellular signaling pathways such as those involving the mitogen-activated protein kinase (MAP kinase) (Dhillon et al., 2007, Oncogene 26: 3279-3290) and the phosphatidylinositol 3-kinase (PI3 kinase). While activation of these pathways has been shown to increase cell proliferation and inhibit apoptosis, inhibition of signaling mediated by HER family members by either small molecule inhibitors or monoclonal antibodies has been shown to inhibit cell proliferation and promote apoptosis (Prenzel et al., 2001, Endocr. Relat. Cancer 8: 11-31).

Heparin-binding epidermal growth factor-like growth factor (HB-EGF) is a 22 kDa, O-glycosylated protein (Higahiyama et al., 1992, J Biol Chem 267: 6205-6212). In its mature form, HB-EGF binds to and activates the EGF receptor and HER4 (Elenius et al., 1997, EMBO 16:1268-1278). HB-EGF is the key mediator of G-protein coupled receptor (GPCR) induced cell proliferation via a process called triple-membrane passing signaling (TMPS) (Prenzel et al., 1999, Nature 402:884-888, review in Fischer et al. 2003, Biochem. Soc. Trans. 31:1203-1208). It has been shown that HB-EGF promotes cellular proliferation as well as angiogenesis (Zushi et al., 1997, Int J Cancer 73:917-923; Abramovitch et al., 1998, FEBS letters 425:441-447). HB-EGF also has been demonstrated to play a key role in a number of cancers, i.e., it has been linked to the aggressive behavior of ovarian tumors (Tanaka et al., 2005, Clin. Cancer Res. 11:4783-4792). Moreover, HB-EGF is essential for xenograft tumor formation by ovarian cancer cell lines. Over-expression of HB-EGF (wild type or a secreted form) accelerates tumor formation in SKOV3 and RMG-1 cells. Knockdown of endogenous HB-EGF using siRNA, yet, abolished or delayed tumor formation by SKOV3 and RMG-1 cells. Miyamoto, 2004, Cancer Res. 64:5720. As suggested by the above evidence, inhibition of HB-EGF expression or activity may inhibit tumor formation.

Similarly, HB-EGF is a marker of poor prognosis in some cancers, including human bladder cancers (Thogersen et al., 2001, Cancer Res. 61:6227-6233). In vitro studies indicate that human EJ bladder cells that were engineered to express HB-EGF (wild type, soluble or non-cleavable) exhibit an increase in growth, anchorage independent growth, and production of VEGF, and enhanced migration. When these HB-EGF-expressing EJ bladder cells were transplanted into nude mice, an increase in tumor formation, size and density of blood vessels was observed in those tumors. (Ongusaha, 2004, Cancer Res. 64:5283-5290).

WO 2009/040134 discloses antigen-binding proteins that bind HB-EGF. The described proteins have been demonstrated to bind to several epitopes of HB-EGF, in particular human HB-EGF. As demonstrated in WO 2009/040134, the ability of HB-EGF to bind to its cognate receptors is reduced or inhibited. As a consequence, the antigen-binding proteins are capable of inhibiting the activity of HB-EGF. Furthermore, WO 2009/040134 suggests the use of the antigen-binding properties for the diagnosis and treatment of proliferative disorders including various types of cancer.

HB-EGF is produced by various tumor cells and acts as an autocrine tumor growth factor. Davis-Fleischer et al., 1998, Front Biosci. 3:288-299; Iwamoto & Mekada, 2000, Cytokine Growth Factor Rev. 11:335-344. HB-EGF has a strong affinity for heparin which can increase the biological activity of HB-EGF. HB-EGF is produced as a transmembrane protein which is proteolytically cleaved by metalloproteinases to yield the mature soluble form of the growth factor.

HB-EGF was first identified from supernatants of cultured human macrophages in a soluble, secreted form. On human cells, the precursor proHB-EGF, acts as the diphtheria toxin receptor. Various cell types, including epithelial cells, keratinocytes, monocytes, mesangial cells, lymphoid cells, and skeletal muscle cells, produce HB-EGF. It is a potent mitogen and chemotactic factor for epithelial cells, fibroblasts, smooth muscle cells and various human cancer cells.

The transmembrane form of HB-EGF is synthesized by many cell types as a 208-amino acid transmembrane precursor (tm-HB-EGF) containing EGF, heparin-binding, transmembrane, and cytoplasmic domains. The extracellular domain can be released as a 12- to 22-kDa soluble form of HB-EGF (sol-HB-EGF) through the action of metalloproteinases, which is regulated by different G protein-coupled receptors (GPCRs) or tumor promoters such as tetradecanoyl phorbol acetate (TPA). Typically, a substantial amount of transmembrane HB-EGF precursor remains uncleaved on the cell surface.

Both tm-HB-EGF and sol-HB-EGF are biologically active. The biological functions of both sol- and tm-HB-EGF are mediated by the EGF receptor (EGFR; HER1) and ErbB4 (HER4). Activation of these types of these receptors is believed to occur as a consequence of ligand-induced receptor homo- or hetero-dimerization. Upon activation, the EGF receptor has been demonstrated to increase cell growth, increase cell motility, inhibit apoptosis and increase cellular transformation.

EGFR-dependent signaling pathways can be transactivated upon stimulation of G-protein-coupled receptors (GPCR). Ligand activation of heterotrimeric G proteins by interaction with a GPCR results in an intracellular signal that induces the extracellular activity of a transmembrane metalloproteinase. Ligands that activate the GPCR pathway include LPA (lysophosphatidic acid), thrombin, carbachol, bombesin, and endothelin. Such activation leads to extracellular processing of a transmembrane growth factor precursor and release of the mature factor which, directly or through the proteoglycan matrix, interacts with the ectodomain of EGFR and activates it through tyrosine phosphorylation. See, Prenzel et al., 1999, Nature 402:884-888. Thus, HB-EGF is a component of a triple membrane-passing signal (TMPS) mechanism whereby a GPCR activates a membrane-bound metalloproteinase, which cleaves proHB-EGF to release the soluble growth factor, which subsequently activates the EGF receptor. EGFR transactivation has been linked to various disease states such as cardiac hypertrophy (reviewed in Shah B H, Catt K J. Trends Pharmacol Sci. 2003 May; 24(5):239-244), vascular remodeling (reviewed in Eguchi et al., 2003, Biochem Soc Trans. 2003 December; 31(Pt 6):1198-202.) and cancer (reviewed in Fischer et al., 2003, supra).

Sequences for HB-EGF proteins and nucleic acids encoding those proteins are available to one of skill in the art. For example, such HB-EGF sequences can be found in the database provided by the National Center for Biotechnology Information (NCBI) (see, http://www.ncbi.nlm.nih.gov/). One example of a sequence for a HB-EGF is the amino acid sequence at NCBI accession numbers NM 001945 and NP_{—}001936 (gi:4503413).

HB-EGF interacts with and activates the epidermal growth factor receptor (EGFR). EGFR is a 170 kDa transmembrane glycoprotein consisting of an extracellular ligand-binding domain, a transmembrane region and an intracellular domain with tyrosine kinase activity. Binding of growth factors to the EGFR results in internalization of the ligand-receptor complex, autophosphorylation of the receptor and other protein substrates, leading ultimately to DNA synthesis and cell division. The external ligand binding domain is not only stimulated by HB-EGF, but also by EGF, TGFα and amphiregulin (AR).

Overexpression of the EGFR is often accompanied by the co-expression of EGF-like growth factors, suggesting that an autocrine pathway for control of growth may play a major part in the progression of tumors. It is now widely believed that this is a mechanism by which tumor cells can escape normal physiological control.

The object of the present application was to provide an more effective treatment of proliferative diseases, in particular cancer. The inventors surprisingly found that several types of proliferative disorders that show an elevated HB-EGF expression at the same time also express EGFR. It has been shown previously that high EGFR ligand concentrations such as HB-EGF can circumvent the effectiveness of human epidermal growth factor receptor (HER family targeted agents) (Ritter et al., 2007; Montero et al., 2008).

Recently, it has been suggested that up-regulation of EGF-like ligands during cetuximab monotherapy compensates for EGFR inhibition (Tabernero et al., 2010). Similarly, combining different anti-HER therapeutics has been shown to enhance responses and overcome acquired resistance in tumors (Storniolo et al., 2008). It appears that functional cooperation among HER and EGF family members play an important role in acquired resistance to anti-HER therapeutics (Wheeler et al., 2008; Motoyama et al., 2002).

Starting from these observations, the inventors of the present invention developed a novel method for treating proliferative disorders. They found that a combined inhibition of HB-EGF and EGFR could be used in those indications.

Accordingly, a first embodiment of the present invention is a composition comprising an antigen-binding protein that binds HB-EGF and an EGFR tyrosine kinase inhibitor for use in the prevention or treatment of proliferative diseases. In particular, the composition is suitable for use in the prevention or treatment of a hyperproliferative disease associated with expression of HB-EGF and/or EGFR. Advantages could be observed in particular in the treatment of hyperproliferative diseases associated with expression of both HB-EGF and EGFR.

The combination of an antigen-binding protein that binds HB-EGF and an EGFR tyrosine kinase inhibitor attacks several steps in the development of proliferative diseases, in particular tumours and other cancerous conditions including signalling events that control cell proliferation, angiogenesis and cell migration associated with the spread and development of metastatic cancer. Such multi-facetic interaction is highly beneficial for controlling and inhibiting the process by which cancer develops. Furthermore, cancers at any stage of progression (e.g., primary, metastatic and recurrent cancers) can be treated.

For example, cancers that can be treated by the claimed compositions include solid mammalian tumors as well as hematological malignancies. Solid mammalian tumors include cancers in children such as, for example, germ cell tumors, soft tissue sarcomas, primary brain tumors, neuroblastoma, nephroblastoma and carcinoma, in particular squamous carcinoma and epithelial carcinoma. Solid mammalian tumors may also include adult cancers such as, for example, tumors of unknown origin, primary brain cancer in adults, tumors of the pituitary gland, lip, oral cavity, Nasopharynx, larynx, maxillary sinus, Ethmoid sinus, salivary glands, thyroid gland (including para thyroid glands and carcinoid), esophagus, stomach, pancreas, small intestine, colon, rectum, anal canal, liver, gallbladder, extra hepatic bile ducts, ampulla of vater, carcinoid, endocrine tumors of gastro-entero-hepatic system, pheochromocytoma and paraganglioma, adrenal glands, lung, pleura, mediastinum, thymus, tumors of bone and soft tissue, skin tumors of lip, eyelid, external ear, other unspecified parts of the face, scalp and neck, trunk, upper limpb and shoulder, lower limb and hip, vulva, penis, scrotum, breast tumors, gynecological tumors of vulva, vagina, cervix uteri, corpus uteri, ovary, fallopian tube, gestational and trophoblastic tumors, penis, prostate, testis, kidney, renal pelvis and ureter, urinary bladder, urethra, ophthalmic tumors of eyelid, conjunctiva, uvea, retina, orbit and lacrimal gland. Hematological malignancies include childhood, for example, leukemia and lymphomas, acute and chronic leukemia (AML, ANLL, ALL, CML, MDS), Hodgkin's disease, B-Cell, T-Cell, large cell, follicular, indolent/low grade, aggressive/high grade lymphomas of lymphocytic and cutaneous origin, plasma cell neoplasm and cancers associated with AIDS.

In addition, the compositions described herein may also be used to treat cancerous conditions or neoplasia disorders, which include, for example, adenoma, tubulovillous adenoma, villous adenoma, angiofibroma, atypical proliferating mucinous neoplasias, Brenner tumor, carcinoid, cavernous hemangioma, cellular leiomyoma, chorangioma, congenital mesoblastic nephroma, mucinous cystadenoma, serous cystadenoma, dermoid, desmoid, fibroadenoma, fibroma, fibrothecoma, follicular adenoma, ganglioneuroma, giant cell tumor, granular cell tumor, granulosa cell tumor, hemangioma, intraductal papilloma, islet cell tumor, leiomyoma, lipoma, luteoma, meningioma, mole, myelolipoma, myxoma, neurofibroma, nevus, osteochondroma, pheochromocytoma, polyposis, schwannoma, serous cystadenoma, struma ovarii, synovial chrondromatosis, benign thymoma.

Further examples of the types of cancers that can be treated with the compositions as described herein may be found, for example, from the American Cancer Society (www.cancer.org), or from Wilson et al. (1991) Harrison's Principles of Internal Medicine, 12^{th} Edition, McGraw-Hill, Inc.

Therefore, the compositions as described herein can be used to treat and/or prevent cancer, cancerous conditions, tumour growth, metastases of cancer cells, angiogenetic processes and/or neoplastic disorders. Thus, these compositions provide a method of treating or preventing cancer in a subject that involves administering to this subject an effective amount of a composition comprising one or more of the antigen-binding proteins that bind HB-EGF and one or more EGFR tyrosine kinase inhibitors.

A high proportion of solid tumor diseases are often characterized by tumor angiogenesis, the excessive growth of (abnormal) vessels in the tumor tissue mediated by growth factors (i.e., VEGF) and other factors (i.e., HB-EGF). Targeting HB-EGF through a HB-EGF-specific antigen binding protein could prevent the formation of new vessels and therefore limit the expansion of existing tumors and the development of new tumors (i.e., metastases).

Besides its role as a mitogenic and pro-invasive ligand several studies have substantiated the picture of HB-EGF as an important regulator of angiogenic processes in cancer. Thus, the compositions as described herein can be used for treating diseases associated with or caused by angiogenesis, e.g., cancerous or non-cancerous diseases.

In a further aspect the compositions as described herein can be used for treating disorders associated with or accompanied by a disturbed, e.g., pathologically enhanced growth factor receptor activation. In another aspect this enhanced growth factor receptor activation may be associated with or caused by a pathological increase in the activity of a G-protein and/or a G-protein-coupled receptor. It should be noted that disorders that are associated with or accompanied by a disturbed, e.g., pathologically enhanced growth factor receptor activation and which are associated with or caused by a pathological increase in the activity of a G-protein and/or a G-protein-coupled receptor, can be delimited from other disorders characterised by an enhanced activity of growth factor receptor activation in that a transactivation of the growth-factor receptor via G-protein-coupled receptor takes place.

According to a most preferred embodiment of the present invention, the composition comprising an antigen-binding protein that binds HB-EGF and an EGFR tyrosine kinase inhibitor is used for the prevention or treatment of a cancer expressing or overexpressing HB-EGF and EGFR. Said cancer may in particular be selected from the group of non-small cell lung cancer, pancreatic cancer, breast cancer, gastrointestinal cancer, prostate cancer, ovarian cancer, stomach cancer, endometrial cancer, salivary gland cancer, lung cancer, kidney cancer, colon cancer, colorectal cancer, thyroid cancer, bladder cancer, glioma, melanoma, carcinoma, in particular epithelial or squamous carcinoma and hepatocellular carcinoma.

During their studies the inventors of the present application surprisingly found that a synergistic effect can be shown for a combination of an antigen-binding protein that binds HB-EGF with Erlotinib. The synergistic effects that could be observed for this specific combination exceeded all expectations. The results are much better than those for the combination with other EGFR tyrosine kinase inhibitors. Erlotinib is an EGFR inhibitor that specifically targets the epidermal growth factor receptor (EGFR) tyrosine kinase. It binds in a reversible fashion to the adenosine triphosphate (ATP) binding site of the receptor. Good results were also obtained for a combination of an antigen-binding protein that binds HB-EGF and gefitinib.

Antigen-binding proteins that bind HB-EGF for use in the composition as described herein are preferably antibodies or fragments thereof, in particular, monoclonal antibodies, polyclonal antibodies, recombinant antibodies, human antibodies, humanized antibodies, chimeric antibodies, multi-specific antibodies or fragments thereof. A fragment may, e.g., be a Fab fragment, a Fab′ fragment, a F(ab′)₂ fragment, a Fv fragment, a diabody or a single-chain antibody molecule.

Preferably, the antigen-binding protein is a human antibody or a humanized antibody. Administration of these human or humanized antibodies reduces the probability of negative side effects. Moreover, these antibodies are stable in vivo, e.g., because they are recognized as normal human products, thereby minimizing the risk of immune system responses. Moreover, these antibodies are not prone to proteolytic destruction, improving their circulating half-life. Hence, the compositions as described herein have an excellent half-life in vivo so that administration in humans is comparatively infrequent. Such as prolonged duration of action may allow for less frequent and more convenient dosing schedules by alternate parenteral routes such as subcutaneous or intramuscular injection.

For use in the presently described compositions, monoclonal antibodies are preferred. In principle, the antibodies may be of any type with IgG1, IgG2, IgG3 or IgG4 type being preferred.

In particular, the antigen-binding proteins disclosed in WO 2009/040134 may be used as antigen-binding proteins which bind HB-EGF.

The isolated antigen-binding proteins are preferably characterised in terms of their complementarity-determining regions (CDR). According to a preferred embodiment of the invention, the antigen-binding protein comprises
A) one or more light chain complementary determining regions (CDRLs) selected from the group consisting of:
   (i) a CDRL1 selected from the group consisting of SEQ ID NOs: 96-124;
   (ii) a CDRL2 selected from the group consisting of SEQ ID Nos:125-140;
   (iii) a CDRL3 selected from the group consisting of SEQ ID NOs:141-181; and
   (iv) a CDRL of (i), (ii) or (iii) that contains one or more amino acid substitutions, deletions or insertions of no more than four amino acids; and/or
B) one or more heavy chain complementary determining regions (CDRHs) selected from the group consisting of:
   (i) a CDRH1 selected from the group consisting of SEQ ID Nos:182-206;
   (ii) a CDRH2 selected from the group consisting of SEQ ID Nos:207-238;
   (iii) a CDRH3 selected from the group consisting of SEQ ID NOs:239-279; and
   (iv) a CDRH of (i), (ii) or (iii) that contains one or more amino acid substitutions, deletions or insertions of no more than four amino acids.

Preferably, the antigen-binding protein comprises at least two CDRLs of A) and/or at least two CDRHs of B).

In a particularly preferred embodiment, the antigen-binding protein comprises
A) a CDRL1 of SEQ ID NOs:96-124, a CDRL2 of SEQ ID NOs:125-140, and a CDRL3 of SEQ ID NOs:141-181, and/or
B) a CDRH1 of SEQ ID NOs:182-206, a CDRH2 of SEQ ID NOs:207-238, and a CDRH3 of SEQ ID Nos:239-279.

Alternatively, it is also possible to use an antigen-binding protein comprising
A) one or more CDRLs selected from the group consisting of:
   (i) a CDRL1 with at least 90% sequence identity to SEQ ID NOs: 96-124;
   (ii) a CDRL2 with at least 90% sequence identity to SEQ ID NOs:125-140; and
   (iii) a CDRL3 with at least 90% sequence identity to SEQ ID Nos:141-181;
B) one or more CDRHs selected from the group consisting of:
   (i) a CDRH 1 with at least 90% sequence identity to SEQ ID NOs:182-206;
   (ii) a CDRH2 with at least 90% sequence identity to SEQ ID NOs:207-238; and
   (iii) a CDRH3 with at least 90% sequence identity to SEQ ID NOs:239-279; or
C) one or more light chain CDRLs of A) and one or more heavy chain CDRHs of B).

Another possibility of characterising the antigen-binding proteins which bind HB-EGF is via the light chain and heavy chain variable regions (VL and VH). Preferably, the antigen-binding protein comprises a light chain variable region (VL) having at least 80% sequence identity with an amino acid sequence selected from the group consisting of SEQ ID NOs: 3-50 and/or a heavy chain variable region (VH) having at least 80% sequence identity with an amino acid sequence selected from the group consisting of SEQ ID NOs: 51-95. Higher sequence identities are even more preferred. For example, preferred antigen-binding proteins that bind HB-EGFR comprise a VL having at least 90% sequence identity with the amino acid sequence of SEQ ID NOs: 3-50 and/or a VH having at least 90% sequence identity with an amino acid sequence selected from the group consisting of SEQ ID NO: 51-95. Most preferably, the VL is selected from the group consisting of SEQ ID NO: 3-50 and/or the VH is selected from the group consisting of SEQ ID NO: 51-95.

According to a preferred embodiment of the present invention, the antigen-binding protein that binds HB-EGF is coupled to an effector group. Examples of effector groups are radioisotopes, radionuclides, toxins, therapeutic groups or chemotherapeutic groups. Therapeutic or chemotherapeutic groups may, e.g., be calicheamicin, auristatin-PE, geldanamycin, maytanasine or derivatives thereof.

Also a subject-matter of the present invention are pharmaceutical compositions including the described combination of an antigen-binding protein that binds HB-EGF and an EGFR tyrosine kinase inhibitor. Therapeutic compositions generally are placed into a container having a sterile access port, e.g., an intervenous solution bag or vial having an adaptor that allows retrieval of the formulation such as a stopper pierceable by a hydrodermic injection needle.

The route of administration of the compositions are in accord with known methods, e.g., injection or infusion by intravenous, subcutaneous, intradermal, intraperitoneal, intracerebral, intramuscular, intraocular, intraarterial, intrathecal, intravesical, intra-cavernous, inhalation, intralesional routes, or by sustained release systems as noted below. In some embodiments, the compositions as described herein are administered continuously by infusion or by bolus injection.

The compositions as described herein can be prepared in a mixture with a pharmaceutically acceptable carrier. This therapeutic composition can be administered intravenously or through the nose or lung, preferably as a liquid or powder aerosol (lyophilized). The composition may also be administered parenterally or subcutaneously as desired. When administered systemically, the therapeutic composition should be sterile, pyrogen-free and in a parenterally acceptable solution with consideration for what are physiologically acceptable pH values, isotonicity, and stability. These conditions are known to those skilled in the art. Briefly, dosage formulations of the compounds described herein are prepared for storage or administration by mixing the compound having the desired degree of purity with physiologically acceptable carriers, excipients, or stabilizers. Such materials are non-toxic to the recipients at the dosages and concentrations employed, and include buffers such as tris HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as Tween, Pluronics or polyethyleneglycol. Sterile compositions for injection can be formulated according to conventional pharmaceutical practice as described in Remington: The Science and Practice of Pharmacy (20th ed, Lippincott Williams & Wilkens Publishers (2003)). For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., 1981, J. Biomed Mater. Res. 15:167-277 and Langer, 1982, Chem. Tech. 12:98-105, or poly(vinylalcohol)), polylactides (U.S. Pat. No. 3,773,919, EP 58,481), copolymers of L-glutamic acid and gamma ethyl-L-glutamate (Sidman et al., 1983, Biopolymers 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals suspended in suitable formulations capable of maintaining crystals in suspension. These preparations when injected subcutaneously or intraperitoneally can produce a sustained release effect. Other compositions also include liposomally entrapped antibodies. Liposomes containing such antibodies are prepared by methods known per se: U.S. Pat. No. DE 3,218,121; Epstein et al., 1985, Proc. Natl. Acad. Sci. USA 82:3688-3692; Hwang et al., 1980, Proc. Natl. Acad. Sci. USA 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the formulation for a given patient will be determined by the attending physician taking into consideration various factors known to modify the action of drugs including severity and type of disease, body weight, sex, diet, time and route of administration, other medications and other relevant clinical factors. Therapeutically effective dosages may be determined by either in vitro or in vivo methods.

An effective amount of the compositions, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.001 mg/kg to up to 100 mg/kg or more, depending on the factors mentioned above. Typically, the clinician will administer the composition until a dosage is reached that achieves the desired effect. The progress of this therapy is easily monitored by conventional assays or as described herein.

It will be appreciated that administration of therapeutic entities in accordance with the compositions and methods herein will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies involving the compositions as described herein, provided that the active ingredients in the formulation are not inactivated by the formulation and the formulation is physiologically compatible and tolerable with the route of administration. See, also Baldrick P. “Pharmaceutical excipient development: the need for preclinical guidance,” 2000, Regul. Toxicol. Pharmacol. 32:210-218; Wang, “Lyophilization and development of solid protein pharmaceuticals,” 2000, Int. J. Pharm. 203:1-60; Charman W N “Lipids, lipophilic drugs, and oral drug delivery-some emerging concepts,” J. Pharm. Sci 0.89:967-978; Powell et al., 1998, “Compendium of excipients for parenteral formulations,” PDA J. Pharm. Sci. Technol. 52:238-311 and the citations therein for additional information related to formulations, excipients and carriers well known to pharmaceutical chemists.

The following figures and examples including the experiments conducted and results achieved are provided for illustrative purposes only and are not to be construed as limiting the teachings of the present invention.

### FIGURES

FIG. 1: Comparison of the tumour growth observed for control, monotherapy with U2-39 and combination therapy with U2-39 and Erlotinib.

### EXAMPLES

### 1. Combination Treatment of U2-39 with Erlotinib—In Vivo Ovarian Xenograft Model

U2-39 is an anti-HB-EGF antibody comprising a CDRL1 with SEQ ID NO: 114, a CDRL2 with SEQ ID NO: 132, a CDRL3 with SEQ ID NO: 164, a CDRH1 with SEQ ID NO: 199, a CDRH2 with SEQ ID NO: 230 and a CDRH3 with SEQ ID NO: 268. The light chain variable region of U2-39 is of SEQ ID NO: 30 and the heavy chain variable region VH of U2-39 is of SEQ ID NO: 81. The complete light chain amino acid sequence is shown in SEQ ID NO: 1, while the heavy chain amino acid sequence is shown in SEQ ID NO: 2.

In order to evaluate the anti-tumor efficacy of the antibody of invention administered as a monotherapy or in combination with Erlotinib, an ovarian cancer xenograft study was conducted.

The human ovarian adenocarcinoma cell line EFO27 was genetically engineered to overexpress HB-EGF. The clone EFO27-CI58 was chosen for xenograft studies in SCID mice. 3×106 EFO27-CI58 cells in 100 μl PBS/Matrigel (1:1) were injected subcutaneously into the left flank of 7-8 week old female CB-17 SCID mice (approximate weight, 30 g). On day 16, after mean tumor volumes had reached approximately 550 mm3, 60 mice were randomized into 5 groups with 12 animals each. On day 17, treatment with U2-39, cetuximab (Erbitux®), Erlotinib (Tarceva®), vehicle control, PBS, or a combination of U2-39 and Erlotinib was initiated. PBS (10 ml/kg), U2-39 (25 mg/kg) and cetuximab (25 mg/kg) were administered intravenously (i.v.) on days 17, 20 and 23 (1, 4 and 7 days after randomization), whereas erlotinib (60 mg/kg) was administered once daily orally (p.o.) from day 17 today 24 either alone or in combination with U2-39. Prior to each treatment on days 17, 20 and 23, tumor volumes were determined by calliper measurement. Following calliper measurement, tumor size was calculated according to the formula W2×L/2 with L=length and W=the perpendicular width of the tumor.

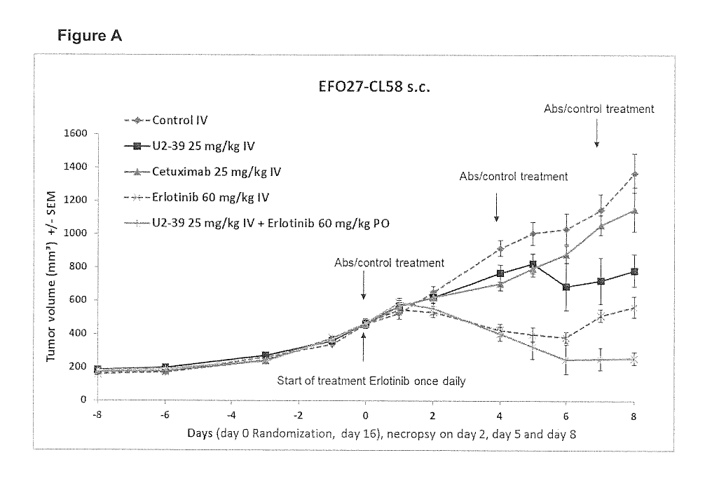

FIG. 1 shows that U2-39 administered three times as monotherapy significantly reduces EFO27-CL58 tumor growth compared to the vehicle control and cetuximab (Erbitux®) monotherapy even though tumor treatment was initiated after after mean tumor volumes had already reached are large volume (˜550 mm3).

It was also demonstrated that a combination of U2-39 with Erlotinib (Tarceva®) led to a stronger tumor reduction during the administration period than treatment with Erlotinib alone.

1.-24. (canceled)

25. A composition comprising:
an antigen binding protein that binds HB-EGF; and,
an EGFR tyrosine kinase inhibitor.

26. The composition of claim 25, wherein the EGFR tyrosine kinase inhibitor is erlotinib or gefitinib.

27. The composition of claim 25, wherein the antigen binding protein is a monoclonal antibody, a polyclonal antibody, a recombinant antibody, a human antibody, a humanized antibody, a chimeric antibody, a multispecific antibody, or an antibody fragment thereof.

28. The composition of claim 25, wherein the antibody fragment is a Fab fragment, a Fab′ fragment, a F(ab)₂ fragment, a Fv fragment, a diabody, or a single chain antibody molecule.

29. The composition of claim 25, wherein the antigen binding protein is a human antibody.

30. The composition of claim 25, wherein the antigen binding protein is a monoclonal antibody.

31. The composition of claim 25, wherein the antigen binding protein is of the IgG1-, IgG2-, IgG3- or IgG4-type.

32. The composition of claim 25, wherein the antigen binding protein is coupled to an effector group.

33. The composition of claim 32, wherein the effector group is a radioisotope, a radionuclide, a toxin, a therapeutic group, or a chemotherapeutic group.

34. The composition of claim 33, wherein the therapeutic group or the chemotherapeutic group is calicheamicin, auristatin-PE, geldanamycin, maytanasine, or derivatives thereof.

35. The composition of claim 25, wherein the antigen binding protein comprises:
A) one or more light chain complementary determining regions (CDRLs) selected from the group consisting of:
(i) a CDRL1 selected from the group consisting of SEQ ID NOs: 96-124;
(ii) a CDRL2 selected from the group consisting of SEQ ID NOs: 125-140;
(iii) a CDRL3 selected from the group consisting of SEQ ID NOs: 141-181; and
(iv) a CDRL of (i), (ii) or (iii) that contains one or more amino acid substitutions, deletions or insertions of no more than four amino acids; and/or
B) one or more heavy chain complementary determining regions (CDRHs) selected from the group consisting of:
(i) a CDRH1 selected from the group consisting of SEQ ID Nos: 182-206;
(ii) a CDRH2 selected from the group consisting of SEQ ID NOs: 207-238;
(iii) a CDRH3 selected from the group consisting of SEQ ID NOs: 239-279; and
(iv) a CDRH of (i), (ii) or (iii) that contains one or more amino acid substitutions, deletions or insertions of no more than four amino acids.

36. The composition of claim 35, wherein the antigen binding protein comprises at least two CDRLs from A) and/or at least two CDRHs from B).

37. The composition of claim 35, wherein the antigen binding protein comprises:
A) a CDRL1 of SEQ ID NOs: 96-124, a CDRL2 of SEQ ID NOs: 125-140, and a CDRL3 of SEQ ID NOs:141-181; and/or,
B) a CDRH1 of SEQ ID NOs: 182-206, a CDRH2 of SEQ ID NOs: 207-238, and a CDRH3 of SEQ ID Nos: 239-279.

38. The composition of claim 36, wherein said antigen binding protein comprises:
A) a CDRL1 of SEQ ID NOs: 96-124, a CDRL2 of SEQ ID NOs: 125-140, and a CDRL3 of SEQ ID NOs:141-181; and/or,
B) a CDRH1 of SEQ ID NOs: 182-206, a CDRH2 of SEQ ID NOs: 207-238, and a CDRH3 of SEQ ID Nos: 239-279.

39. The composition of claim 35, wherein THE antigen-binding protein comprises:
A) one or more CDRLs selected from the group consisting of:
(i) a CDRL1 with at least 90% sequence identity to SEQ ID NOs: 96-124;
(ii) a CDRL2 with at least 90% sequence identity to SEQ ID NOs: 125-140; and
(iii) a CDRL3 with at least 90% sequence identity to SEQ ID Nos: 141-181; or,
B) one or more CDRHs selected from the group consisting of:
(i) a CDRH 1 with at least 90% sequence identity to SEQ ID NOs: 182-206;
(ii) a CDRH2 with at least 90% sequence identity to SEQ ID NOs: 207-238; and
(iii) a CDRH3 with at least 90% sequence identity to SEQ ID NOs: 239-279; or,
C) one or more light chain CDRLs of A) and one or more heavy chain CDRHs of B).

40. The composition of claim 35, wherein the antigen binding protein comprises a light chain variable region (VL) having at least 80% sequence identity with an amino acid sequence selected from the group consisting of SEQ ID NOs: 3-50, and/or a heavy chain variable region (VH) having at least 80% sequence identity with an amino acid sequence selected from the group consisting of SEQ ID NOs: 51-95.

41. The composition of claim 40, wherein the VL has at least 90% sequence identity with an amino acid sequence selected from the group consisting of SEQ ID NOs: 3-50, and/or the VH has at least 90% sequence identity with an amino acid sequence selected from the group consisting of SEQ ID NOs: 51-95.

42. The composition of claim 40, wherein the VL is selected from the group consisting of SEQ ID NOs: 3-50, and/or the VH is selected from the group consisting of SEQ ID NOs: 51-95.

43. The composition of claim 25 further comprising one or more pharmaceutically acceptable carriers, additives, stabilizers, excipients or a combination thereof.

44. A method for the prevention or treatment of a hyperproliferative disease associated with expression of HB-EGF and/or EGFR in an animal or human comprising administration of the composition of claim 43 to the animal or human in an amount effective to prevent or treat the hyperproliferative disease.

45. The method of claim 44, wherein the hyperproliferative disease is associated with or accompanied by a pathologically enhanced growth factor receptor activation.

46. The method of claim 45, wherein the pathologically enhanced growth factor receptor activation is associated with or caused by a pathological increase in the activity of a G protein and/or a G protein coupled receptor.

47. A method for the prevention or treatment of cancer in an animal or human comprising administration of the composition of claim 43 to the animal or human in an amount effective to prevent or treat the cancer.

48. The method of claim 47, wherein the cancer expresses or overexpresses HB-EGF and/or EGFR.

49. The method of claim 47, wherein the cancer is selected from the group consisting of non-small cell lung cancer, pancreatic cancer, breast cancer, gastrointestinal cancer, prostate cancer, ovarian cancer, stomach cancer, endometrial cancer, salivary gland cancer, lung cancer, kidney cancer, colon cancer, colorectal cancer, thyroid cancer, bladder cancer, glioma, melanoma, carcinoma, in particular epithelial or squamous carcinoma and hepatocellular carcinoma.

50. The method of claim 48, wherein the cancer is selected from the group consisting of non-small cell lung cancer, pancreatic cancer, breast cancer, gastrointestinal cancer, prostate cancer, ovarian cancer, stomach cancer, endometrial cancer, salivary gland cancer, lung cancer, kidney cancer, colon cancer, colorectal cancer, thyroid cancer, bladder cancer, glioma, melanoma, carcinoma, in particular epithelial or squamous carcinoma and hepatocellular carcinoma.
